(19) United States
(12) Patent Application Publication
(10) Pub. No.: US 2019/0269877 A1
(43) Pub. Date: Sep. 5, 2019

(54) Electromagnetic resonant frequency coil ion emitter

(71) Applicant: Dennis Yat Wong, BC (CA)

(72) Inventor: Dennis Yat Wong, BC (CA)

(21) Appl. No.: 15/932,474

(22) Filed: Mar. 5, 2018

(51) Int. Cl. A61M 21/00; A61N 2/00; A61N 2/02; A01M 29/28

(52) U.S. Cl. CPC A61M 21/00; A61N 2/002; A01M 29/16; A01M 29/28; A61M 2021/0055; A61N 2/02

(57) ABSTRACT

An electromagnetic resonant frequency ion coil emitter is disclosed. An embodiment of the invention being comprised of a planar-shaped, platform with metal pins configured in a 14-point star arrangement disposed to receive several layers of a copper wire winding thereon. Other embodiments may include an area-wide device and a hand held device with said coil including gems therein. The invention being connected to a plurality of external, predetermined digital or analog sound frequencies and or wave forms by means of an external amplifier thereinbetween. Upon receiving amplified frequencies, said copper conducting an electromagnetic field and associated positive and negative ions into the shape of a vortex to be received by a user seeking therapeutic benefits. Other area-wide and hand held embodiments of the invention may be utilized for vector control in agricultural and therapeutic settings respectively.

### FIELD OF THE INVENTION

The present invention generally relates to resonant frequencies. More specifically, it relates to a method and system for generating electromagnetic fields from amplified digital frequencies.

### BACKGROUND

Resonant frequency was first developed in the early 1900s by a scientist known as Royal Rife. Royal transmitted various frequency waves with a microscope device and targeted known pathogens. He found that certain pathogens responded to certain frequencies. In the 1950s, researchers began extrapolating these frequencies to treat human diseases and Dr. Abraham Ginsberg successfully treated patients who had various infections and went on to found the Ginsberg Foundation for Medical Research Symposium. In the early 1990s medical researchers began developing portable resonant frequency devices. U.S. Pat. No. 5,402,782, issued Apr. 4, 1995, and U.S. Pat. No. 5,553,610, issued Sep. 10, 1996, both to Robert A. Lodder disclosed a small magnetic, near-infrared radiation and an acoustic wave delivery system for therapeutic purposes. U.S. Pat. No. 4,535,775 disclosed a method of healing bone fractures by applying to electrodes coupled to the skin of a living body in the vicinity of a bone fracture and transmitting an electromagnetic frequency in the range of 20-100 KHZ and a value in the range from about 2 to 10 volts. Several commercially available rife machines are available on the market that deliver electromagnetic frequencies using attachable electrodes, passive electrostatic tubes and hand held cylinders. However, none were found in the prior art that incorporated a coil bound in a multi-point pattern connected to external amplifiers and digital audio frequency files.

### SUMMARY OF THE INVENTION

The device herein disclosed and described provides a solution to the shortcomings in the prior art through the disclosure of a method and system for transmitting resonant frequencies through a copper coil emitter. The device is comprised of a base with conductive poles arranged in a star-like, cylindrical pattern. The poles are wound with copper wire and is connected to digital audio frequency library by means of a powerful amplifier. When activated, audio files are played into the amplifier where they frequencies are amplified and sent to the coil allowing it to emit an electromagnetic vortex field.

An object of the invention is to provide a means to deliver electromagnetic frequencies to a user in a room. When the user plays an audio file into the amplifier, it sends the frequency to the coil and then rotates the signal at the speed of light in a circular fashion creating a frequency pulsed electromagnetic vortex field with a plurality of positive and negative ions that surrounds a user. Crystalline structures such as gems can also be positioned at each star point allowing the stones to generate additional positive ions.

An object of the invention is to provide a means to deliver pinpointed electromagnetic frequencies to a specific portion of a user's body by means of a handheld wand. When the user plays an audio file into the amplifier, it sends the frequency to the coil inside a cylindrical wand and then rotates the signal at the speed of light in a circular fashion creating a frequency pulsed electromagnetic vortex field. Also included in the wand are piezoelectric crystalline structures (gems etc.) that when exposed to sound also contribute to the emission of positive ions that are directed at the user.

Another object of the invention is to provide a means to deliver electromagnetic frequencies to infrastructure to promote wellness, calm the nervous system and provide brainwave entertainment among several occupants. This configuration can also be therapeutic to animals when setup in pens, cages and the like. In this embodiment, coils are placed at each floor of a building. Each coil is connected to a central digital audio and amplifier and exposes occupants to positive ion vortexes.

Another object of the invention is to provide a means to deliver electromagnetic frequencies and positive ions to a farm field to disrupt or scare invasive insects from harming crops. In this embodiment, coils are placed along vulnerable rows and connected to a single digital audio frequency player by a common amplifier that distribute vortexes within the field thereby disrupting insects within the vicinity.

It is briefly noted that upon a reading this disclosure, those skilled in the art will recognize various means for carrying out these intended features of the invention. As such it is to be understood that other methods, applications and systems adapted to the task may be configured to carry out these features and are therefore considered to be within the scope and intent of the present invention, and are anticipated. With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention. As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The objects features, and advantages of the present invention, as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTION OF THE FIGURES

The accompanying drawings, which are incorporated herein and form a part of the specification, illustrate some, but not the only or exclusive, examples of embodiments and/or features.

FIG. 1 shows a perspective view of an area-wide embodiment of the invention.

FIG. 2 shows a perspective view of the invention with its electromagnetic vortex field.

FIG. 3 shows a top view of an embodiment of the invention being used in tandem.

FIG. 4 shows a side view of the invention being used in triplicate.

FIG. 5 shows a side view of the invention interacting with source audio devices and amplifier.

FIG. 6 shows a side view of an area-wide embodiment in an agricultural setting.

FIG. 7 shows a side view of a hand held embodiment of the invention with internal components.

FIG. 8 shows a side view of a hand held embodiment of the invention.

FIG. 9 shows a front view of a handheld embodiment the invention.

FIG. 10 shows a person using the handheld embodiment of the invention.

Other aspects of the present invention shall be more readily understood when considered in conjunction with the accompanying drawings, and the following detailed description, neither of which should be considered limiting.

### DETAILED DESCRIPTION OF FIGURES

In this description, the directional prepositions of up, upwardly, down, downwardly, front, back, top, upper, bottom, lower, left, right and other such terms refer to the device as it is oriented and appears in the drawings and are used for convenience only; they are not intended to be limiting or to imply that the device has to be used or positioned in any particular orientation.

FIG. 1 shows a perspective view of an area-wide embodiment of the invention comprised of a square-shaped base plate 1 being made of a rigid material such as, but not limited to wood, metal and the like. Said base 1 connecting a metal wire coil 5 connected by means of circular-shaped bolts 4. The wire 5 comprised of, but not limited to, a conductive metal such as, but not limited to copper etc. and said bolts 4 made of a rigid metal such as stainless steel aluminum, steel and the like. The sleeves being disposed radially at, but not limited to, 14 points around said base 1. Metal wire coil 5 having at least three levels of windings around said sleeves 4 and connected to audio devices by means of an audio jack therein. A distal end of the invention having a translucent, planar plate made of, but not limited to glass etc. and being connected to said base 1 by means of mounting cylinders 3. Said cylinders made of, but not limited to: a rig material such as metal, plastic and the like. FIG. 1 also showing a crystalline structure such as gems 15 can also be positioned at a point on the coil or said mounting cylinders 3 allowing the stones to generate additional positive and negative ions once exposed to vortex frequencies.

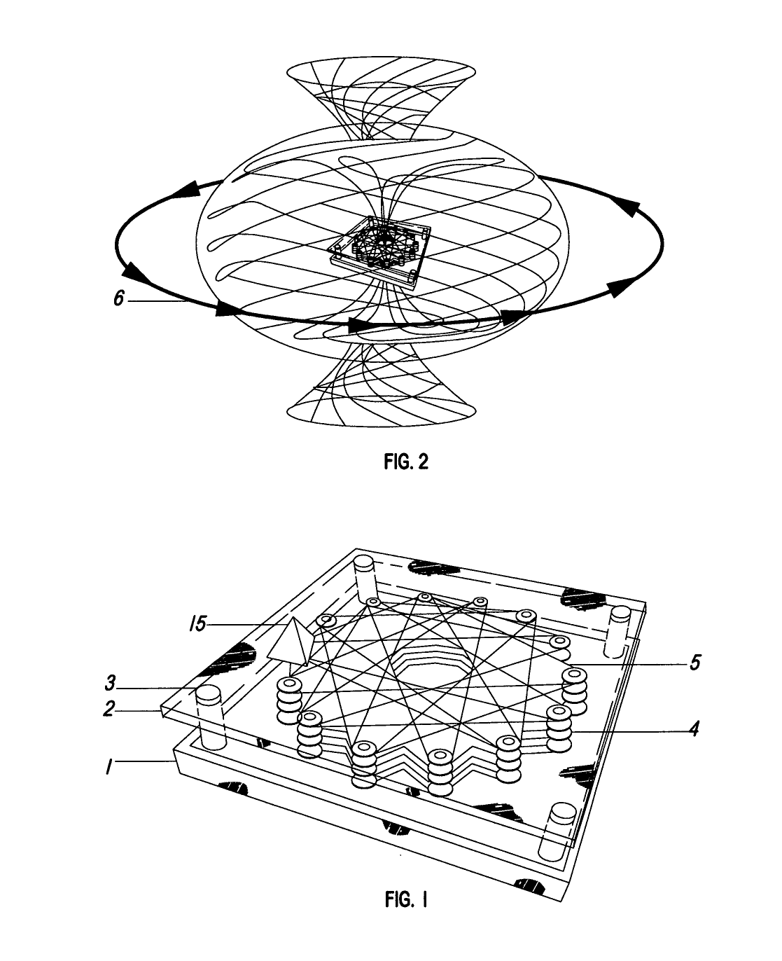

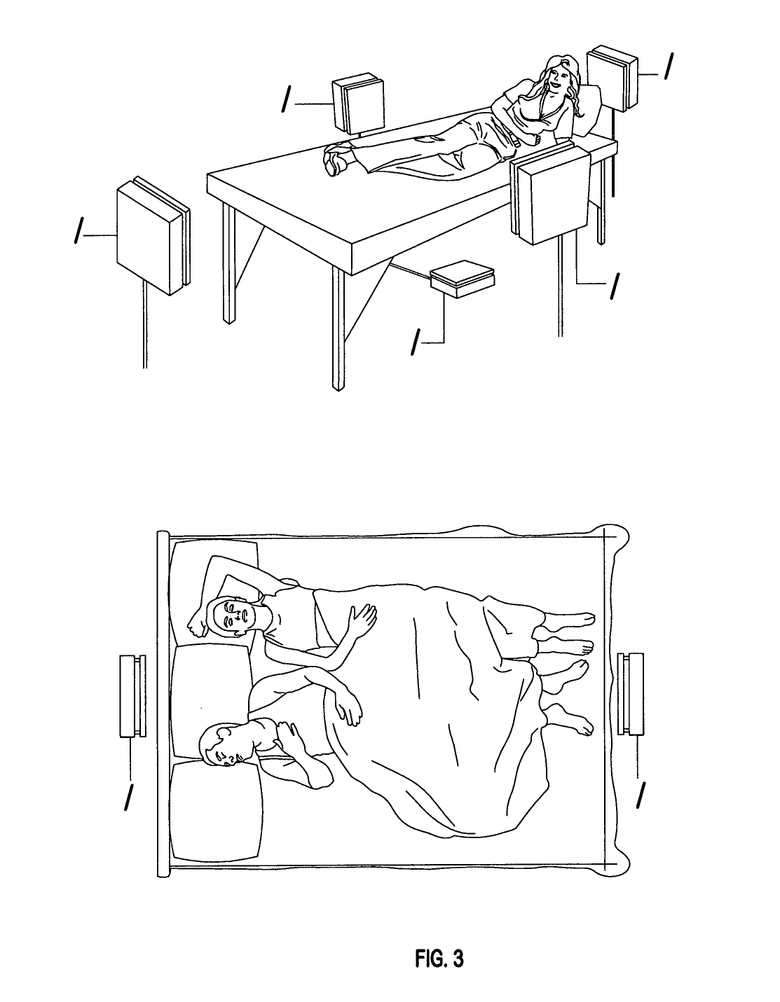

FIG. 2 shows a perspective view of the area-wide embodiment of the invention with its electromagnetic vortex field 6 being emitted from the metal wire coil 5 and rotating a signal at the speed of light in a circular fashion creating a frequency pulsed electromagnetic field vortex field with a plurality of positive ions that surrounds a user.

FIG. 3 shows a top view of an area-wide embodiment of the invention being used in tandem on each side of a pair of users allowing said metal wire coil 5 to emit said electromagnetic vortex field 6 along with ions. FIG. 3 also showing a perspective view of a user having five area-wide embodiments positioned around a user on a message table in five planes.

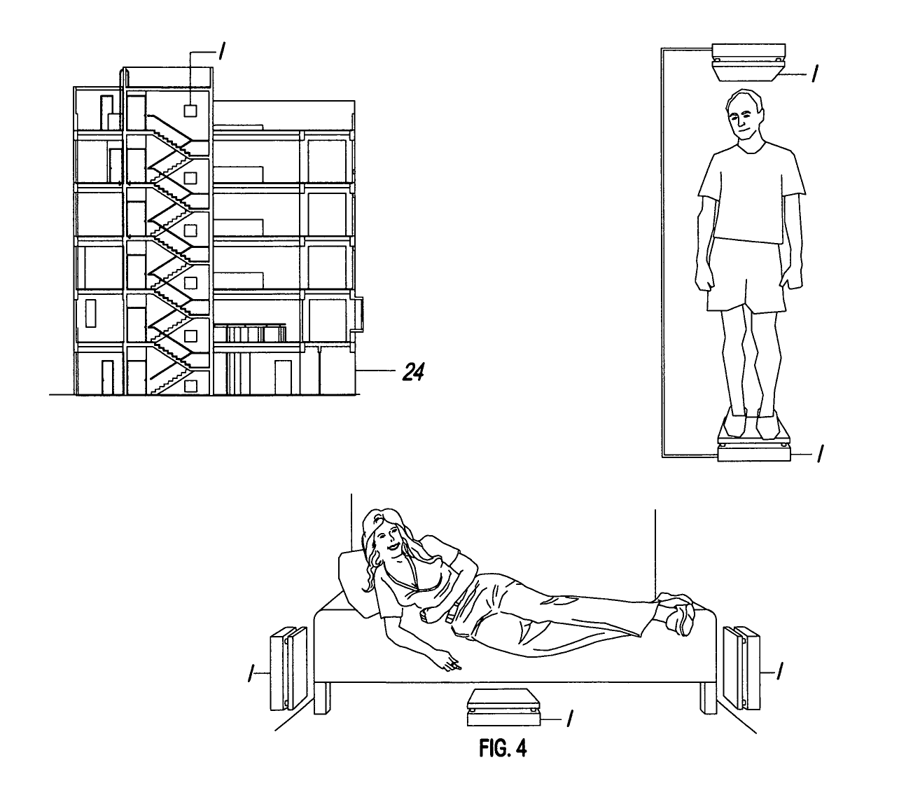

FIG. 4 shows a side view of the area-wide embodiment of the invention being used in triplicate having an invention disposed underneath and beside a user in several directions. The figure also showing the room embodiment being positioned on each floor of a building 24 providing exposure for all occupants therein. FIG. 4 also showing said area-wide embodiment of the invention being used in duplicate around a user in two planes.

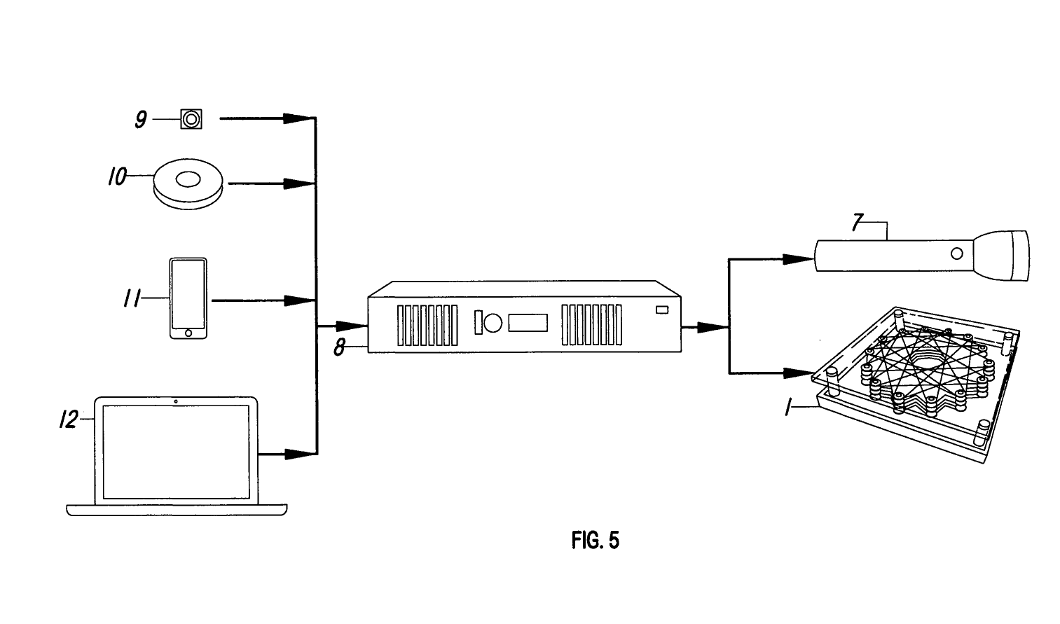

FIG. 5 shows a side view of the area-wide embodiment and handheld embodiment 7 of the invention accepting source audio devices including, but not limited to: an iPOD™ 9, CD player 10, mobile device 11 (such as Android™, Apple™ etc.) and computer (such as Windows™, Apple™ etc.) 12 by means of an amplifier 8. Said amplifier 8 being comprised of, but not limited to: Crown™ XLS 350W, Behringer™ iNuke NU 1000, Qinpu™ A-6500 etc. Said audio being comprised of a plurality of ‘quantum waveform’ frequencies associated known to be associated with mitigating a multitude of maladies such as, but not limited to: Allergies, emotional issues, chronic illnesses etc. Frequencies may include but not be limited to: 3 hz to 1,000 hz at a multitude of incremental frequencies preset by said digital audio devices. Said audio devices being connected to amplifiers and invention embodiments by, but not limited to: USB, RCA and XLR cables and the like.

FIG. 6 shows a side view of an area-wide embodiment in an agricultural setting with area-wide embodiments of the invention being positioned within proximity of plants 14 and sending said electromagnetic vortex field 6 to plants by means of amplifier 8 and main controller 13 and providing growth stimulation to said plants 14.

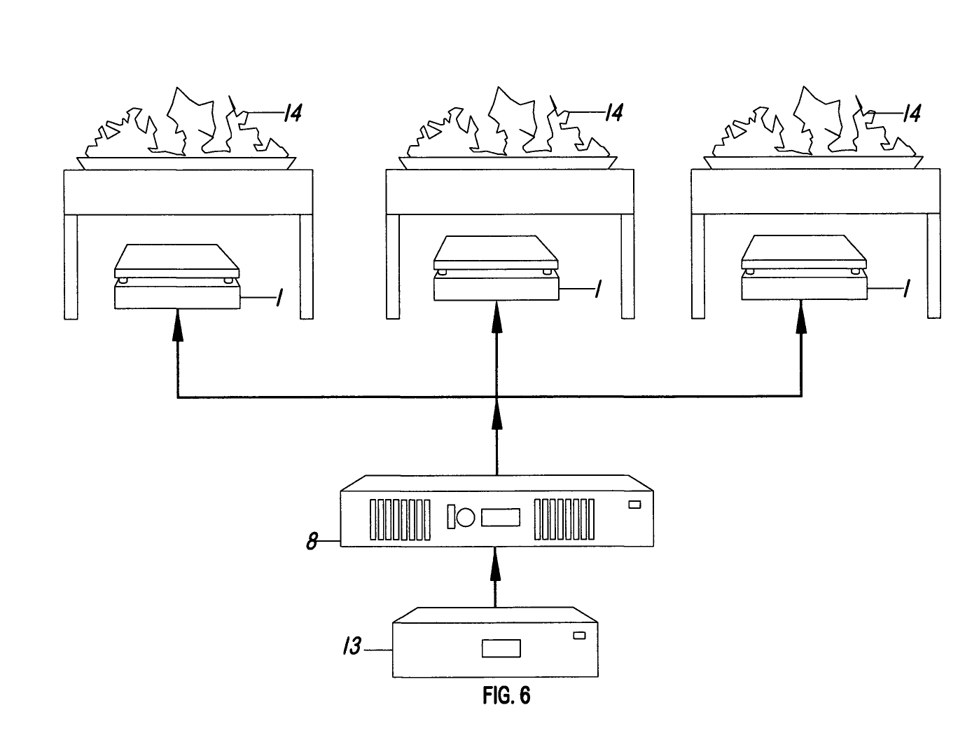

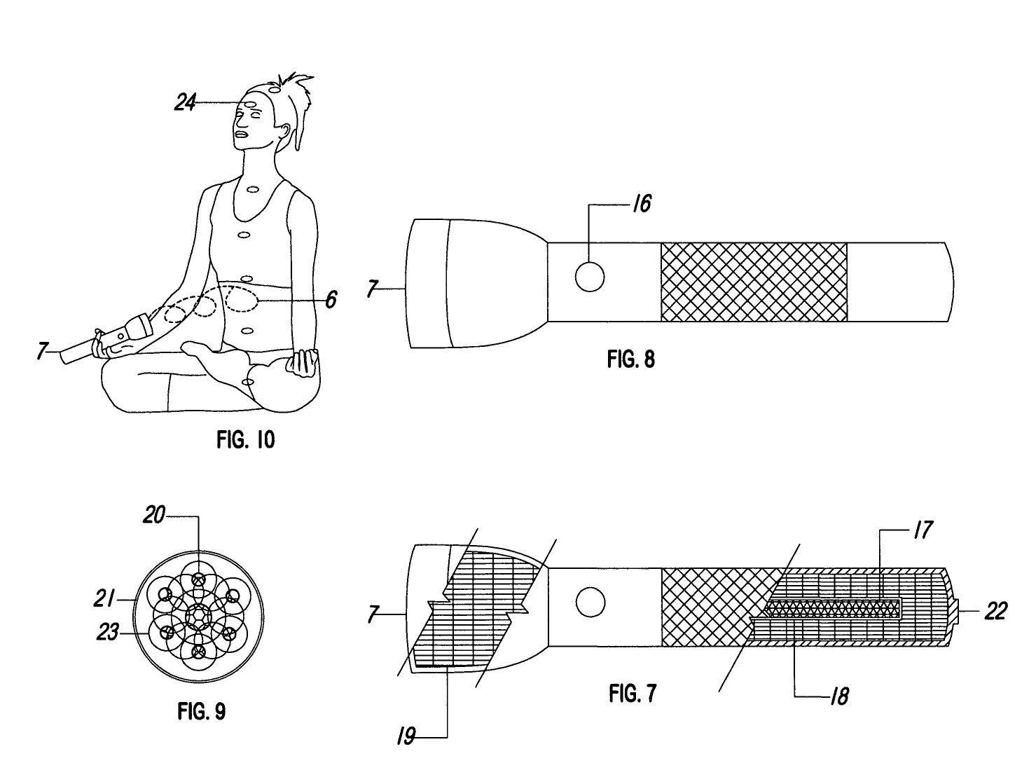

FIG. 7 shows a side view of a hand held embodiment 7 of the invention with internal components comprised of the aforementioned metal windings 19 being configured as a miniature version of the area-waide embodiment and only being wound on six points rather than 14. The embodiment having a stainless steel internal core 18 having geometric windings 17 with at least 33 tourmaline gems. Said gems also being disposed within and around said metal windings 19 and when exposed to said frequency vortex and emitting positive and negative ions from the invention. The handheld embodiment operating similar to the area-wide embodiment having an audio input via an amplifier and audio devices via an audio jack 22 on one end.

FIG. 8 shows a side view of a hand held embodiment of the invention having a cylindrical shell made of but not limited to: 6065 aluminum and having ventilation portal 16 to shed extraneous heat.

FIG. 9 shows a front view of a handheld embodiment of the invention with planar face 21 place made of a crystalline material with a plurality of orifices in a radial pattern therein. Each orifice disposed to receive a crystalline stone 20, such as but not limited to ruby gems etc. Said gems being exposed to said vortex frequencies and emitting positive ions by means of piezometric mechanisms. The aforementioned face plate 21 also having a plurality of elliptical groves therein allowing frequencies from the aforementioned metal windings 19.

FIG. 10 shows a person using the handheld embodiment of the invention being operated by a user during meditation. The invention sending vortex frequencies 6 to user's ‘chakras’ 24 promoting healing.

It is additionally noted and anticipated that although the device is shown in its most simple form, various components and aspects of the device may be differently shaped or slightly modified when forming the invention herein. As such those skilled in the art will appreciate the descriptions and depictions set forth in this disclosure or merely meant to portray examples of preferred modes within the overall scope and intent of the invention, and are not to be considered limiting in any manner. While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention.

What is claimed is:

1. A electromagnetic resonant frequency ion coil emitter comprised of a band of wires around pins in a pattern that receive digital audio signals from an audio device and emitting vortex frequencies and ions. The invention having an area-wide and handheld embodiments and both embodiments.

2. The resonant frequency ion coil emitter of claim 1 providing a means to deliver electromagnetic frequencies and ions to a user within the vicinity.

3. The resonant frequency ion coil emitter of claim 1 providing a means to deliver pinpointed electromagnetic frequencies to a specific portion of a user's body by means of a handheld wand embodiment.

4. The resonant frequency ion coil emitter of claim 1 providing a means to deliver electromagnetic frequencies to infrastructure to promote wellness among several occupants.

5. The resonant frequency ion coil emitter of claim 1 providing a means to deliver electromagnetic frequencies and positive ions to a farm field to disrupt invasive insects from harming crops.
